(19) United States
(12) Patent Application Publication
(10) Pub. No.: US 2021/0057063 A1
(43) Pub. Date: Feb. 25, 2021

(54) EXTRACTING CLINICALLY RELEVANT INFORMATION FROM MEDICAL RECORDS

(71) Applicant: Regents of the University of Minnesota, Minneapolis, MN (US)

(72) Inventors: Christopher Tignanelli, Edina, MN (US); Genevieve Melton-Meaux, Minneapolis, MN (US); Serguei V.S. Pakhomov, Falcon Heights, MN (US); Elizabeth Lindemann, Minneapolis, MN (US); Greg Silverman, St Louis Park, MN (US)

(21) Appl. No.: 17/000,006

(22) Filed: Aug. 21, 2020

Related U.S. Application Data
(60) Provisional application No. 62/891,024, filed on Aug. 23, 2019.

(51) Int. Cl. G16H 10/60; G16H 15/00; G16H 70/20; G16H 50/70; G16H 50/20; G06F 40/289; G10L 15/26; A61B 5/00

(52) U.S. Cl. CPC G16H 10/60; G16H 15/00; G16H 70/20; G16H 50/70; A61B 2505/01; G06F 40/289; G10L 15/26; A61B 5/749; G16H 50/20

(57) ABSTRACT

In some examples, a computing system includes a data repository configured to store a plurality of treatments and a corresponding plurality of clinically relevant phrases; and a computing system comprising processing circuitry configured to receive prehospital data; determine at least one clinically relevant phrase of the plurality of clinically relevant phrases present in the EMS prehospital data; and determine at least one recommended treatment associated with the at least one clinically relevant phrase.

This application claims the benefit of U.S. Provisional Patent Application No. 62/891,024, filed Aug. 23, 2019, the entire content of which is incorporated herein by reference.

### GOVERNMENT INTEREST

This invention was made with government support under UL1TR002494 awarded by National Institutes of Health (NIH) and R01HS024532 awarded by Agency for Healthcare Research and Quality (AHRQ). The government has certain rights in the invention.

### BACKGROUND

Trauma is the leading cause of death for Americans younger than 45 years old. A 2016 National Academies of Sciences Engineering and Medicine report identified that nearly 20% of trauma deaths are preventable, with national efforts aimed at achieving the goal of zero preventable deaths. While significant attention has been focused on improving in-hospital mortality, less attention has been focused on prehospital care.

### SUMMARY

This disclosure describes systems and methods for extracting clinically relevant data from medical records such as emergency medical services (EMS) prehospital data and, in some examples, processing the extracted data to improve prehospital treatment of patients. In some examples, a computing system is configured to apply natural language processing (NLP) and information extraction (IE) to EMS records to determine one or more applied clinical procedures and evaluate the appropriateness of the applied clinical procedures. In other examples, a computing system is configured to apply real-time NLP to EMS audio data, such as data recorded via a microphone in an ambulance. The computing system may then determine, based on the audio data, one or more recommended treatments; and output the recommended treatments for display on an ambulance monitor.

In some examples, a system includes a data repository configured to store a plurality of treatments and a plurality of respective clinically relevant phrases, each of the respective clinically relevant phrases being associated with at least one treatment of the plurality of treatments; and a computing system comprising processing circuitry configured to: receive prehospital data; determine at least one clinically relevant phrase of the plurality of clinically relevant phrases present in the EMS prehospital data; and determine at least one recommended treatment associated with the at least one clinically relevant phrase.

In some examples, a method includes receiving, by processing circuitry, prehospital data; determining, by the processing circuitry, at least one clinically relevant phrase present in the EMS prehospital data; determining, by the processing circuitry, at least one recommended treatment associated with the at least one clinically relevant phrase; and outputting, by the processing circuitry, the at least one recommended treatment.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a block diagram illustrating an example computing system for extracting and evaluating prehospital clinical data from EMS records, in accordance with some techniques of the present disclosure.

FIG. 2 is a decision tree illustrating a set of annotation relationships indicating how schema entities may be semantically connected with each other, in accordance with some example techniques of this disclosure.

FIG. 3 is a flow diagram depicting an example process of extracting and evaluating clinical data from prehospital EMS records, in accordance with some techniques of this disclosure.

FIG. 4 is a flowchart illustrating an example operation in accordance with the present techniques.

FIG. 5 is a block diagram illustrating an example computing system for generating real-time recommended treatments, in accordance with one or more techniques of the present disclosure.

FIG. 6 is a flowchart illustrating an example operation in accordance with the present techniques.

FIG. 7 is a block diagram illustrating an example of various devices that may be configured to implement one or more techniques of the present disclosure.

FIG. 8 is a block diagram illustrating a system including an example medical device configured to perform the techniques of this disclosure.

FIG. 9 is a bar graph depicting example precision and recall of natural-language processing compared with manual review for airway intervention (AI) and chest compression system (CSS), in accordance with the techniques of this disclosure.

### DETAILED DESCRIPTION

Trauma is the leading cause of death in the United States of America (US) and worldwide for people under the age of 45. Unfortunately, there are approximately 6 million traumatic deaths a year. This is significant, as more people die each year from trauma than from malaria, tuberculosis, and HIV/AIDs combined. Additionally, studies have found that nearly 20% of trauma deaths are preventable, with the majority of preventable deaths occurring in the prehospital setting. These deaths may be due to quality gaps in prehospital and intrahospital trauma systems. A key barrier limiting improvement of prehospital patient care is a lack of robust data indicating specific avenues for improvement. While efforts are underway within the USA to develop a national Emergency Medical Services (EMS) database (e.g., NEMSIS 3), many shortfalls exist. For example, participation is voluntary and requires laborious documentation practices or manual data abstraction of EMS reports. Additionally, critical decision-making data present in the unstructured narrative field of EMS run reports is not captured. Finally, the NEMSIS 3 data standard has not yet been adopted by all US states. Thus, there is a critical need to characterize and ultimately improve prehospital EMS documentation. Filling this gap will inform the development of prehospital data standards for documentation. The creation of these standards provides an opportunity to assess and characterize how well trauma elements are currently represented within unstructured prehospital clinical documentation.

Prehospital data includes any patient data obtained prior to arriving at a hospital or clinic, such as data generated by EMS technicians, police, or other sources as to the condition of the patient prior to arriving at the hospital. One example of prehospital data is EMS narrative data, such as an EMS technician's audio recordings and/or written notes, which may provide a rich resource for trauma-management evaluation. Improved prehospital field management could prevent future trauma deaths. However, much variation in both documentation frequency and content of EMS notes currently exists. Natural-language processing (NLP) techniques may be employed by a computer system to leverage and extract data from EMS records and characterize treatment appropriateness efficiently and with high fidelity, ultimately to inform clinical decision-making for improved outcomes for patients, such as motor vehicle crash (MVC) trauma patients.

In general, this disclosure describes systems and methods for processing EMS data, (e.g., extracting clinically relevant information from EMS records), and assessing and/or improving patient treatment based on the extracted information. For example, a system may extract clinically relevant information from EMS or other prehospital records, determine whether any treatment applied to the patient prior to arriving at a hospital or clinic differs from recommended procedures based on the extracted clinically relevant information, and then present those differences. In other examples, a system may extract clinically relevant information from EMS or other prehospital records (e.g., audio and/or textural data), determine recommended treatment based on the extracted clinically relevant information, and then present that recommended treatment to technicians, such as EMS technicians or other user. In this manner, the systems and techniques described herein may provide retrospective analysis of prehospital treatment and/or real-time treatment assistance to medical professionals or other users based on audio and/or textural information associated with the patient.

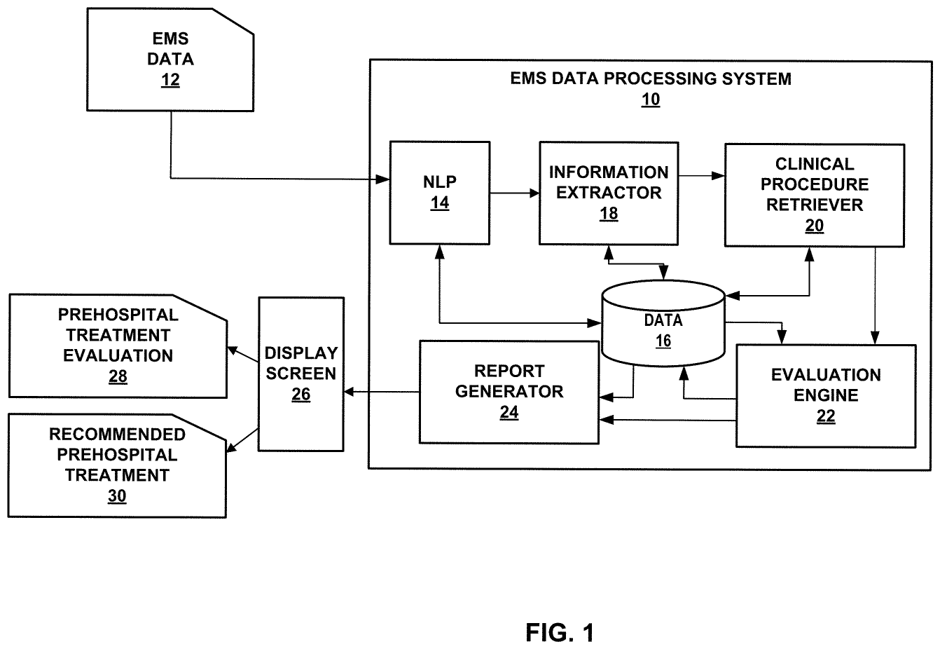

FIG. 1 is a block diagram illustrating an example computing system 10 for extracting and evaluating prehospital clinical procedures from EMS records, in accordance with one or more techniques of the present disclosure. In the example of FIG. 1, system 10 may represent a computing device or computing system, such as a mobile computing device (e.g., a smartphone, a tablet computer, a personal digital assistant, and the like), a desktop computing device, a server system, a distributed computing system (e.g., a “cloud” computing system), or any other device capable of receiving EMS records 12 and performing the techniques described herein.

As further described herein, EMS data processing system 10 is configured to receive data input, for example, in the form of EMS data 12. EMS data 12 may include recorded information regarding an EMS technician's treatment of one or more patients, such as a trauma patient. In some examples, EMS data 12 may include the technician's written textual notes, describing a diagnosis of, and/or a treatment applied to a trauma patient. In other examples, EMS data 12 may include audio data, such as audio recorded by a microphone, of an emergency medical technician (EMT) assessing and/or treating a trauma patient.

EMS data processing system 10 is configured to receive EMS data 12 and then process the data in order to extract clinically relevant information from the data to evaluate and/or improve patient treatment. In some examples, such as the example depicted in FIG. 1, system 10 is configured to extract information indicating a condition of the trauma patient and/or prehospital clinical procedures applied to the patient, and then evaluate (e.g., assess) the appropriateness of the applied clinical procedures. Processing system 10 may include multiple sub-routines 14, 18, 20, 22, and 24. These sub-routines may all be conducted on the same hardware components (e.g., processing circuitry) or on different components in data communication with one another.

Processing system 10 includes one or more sub-routines dedicated to natural language processing (NLP) 14 and information extraction (IE) 18 of EMS data 12. NLP 14 and IE 18 may be configured to identify (e.g., recognize) and categorize one or more key words or phrases from EMS data 12 according to a standardized annotation schema. For instance, in the examples in which EMS data 12 includes recorded audio data, NLP 14 may be configured to first apply a speech-recognition module to EMS data 12 in order to convert the audio data into textual data. NLP 14 and IE 18 may then process the converted text file, or in other examples, the EMT's textual narrative notes, to recognize, identify, annotate, and/or categorize clinically relevant key words and phrases, according to a developed standard keyword schema. The developed keyword schema may help explain and represent EMS vocabulary in order to inform current EMS data standards and terminologies like NEMSIS, and further develop novel NLP/IE systems for extraction of clinically relevant data. In some examples, system 10 may be configured to identify and select either a particular NLP module 12, or particular combination of different NLP modules 12, from among several NLP options, such that IE 18 may more-accurately and/or more-efficiently extract keywords according to a keyword annotation schema.

One example standard keyword schema may be developed according to the following example. EMS reports 12 contain trauma-domain knowledge, vocabulary, and structure. Developing a keyword schema to characterize and represent current EMS documentation practices may help inform data standards, standard terminologies, and NLP/IE techniques, and provide potential tactics to improve documentation practices and processes. The developed schema may further enhance the generation of standards for EMS documentation (e.g., expanding templates for semi-structured data entry and adding clinically relevant structured data entry), and may ultimately have the potential of improving both patient care and research. The schema may utilize motor vehicle crash (MVC) EMS run reports as an initial use case to provide characterization of trauma elements as documented within free-text narrative prehospital documentation, as validated by the NEMSIS 3 data standards. This characterization may help to identify refinements that could be made to existing standard terminologies for trauma elements, and provide insights for future standardization for trauma documentation.

The schema may incorporate annotation and guidelines, for example, using the brat rapid annotation tool (BRAT). Annotations may be performed on the clinical text corpus 12 and gaps may be identified in the annotation guidelines and schema. These gaps in coverage may be addressed and integrated into the annotation guidelines. Data elements may be added iteratively to capture the diverse content of EMS notes 12 and ensure data completeness for analysis. Data completeness may be determined through a manual review of desired data elements within each note. An overlapping set of documents may be annotated to calculate inter-rater reliability between three raters. Additionally, attributes may be captured for each entity to better characterize data elements, specifically: certainty, strength of procedural indication (strong indication, medium indication, mild indication, no indication), passenger status, and quantitative/qualitative descriptive text (exact, inexact quantitative, and inexact qualitative).

Annotation may be conducted at the most-specific-feasible level of detail. Entities may be defined in parent-child relationships. For example, in the sentence, “the patient was in a head-on MVC (motor-vehicle collision),” the text “head-on MVC” may be annotated as the child entity “head-on”, rather than the parent entity “MVC type”.

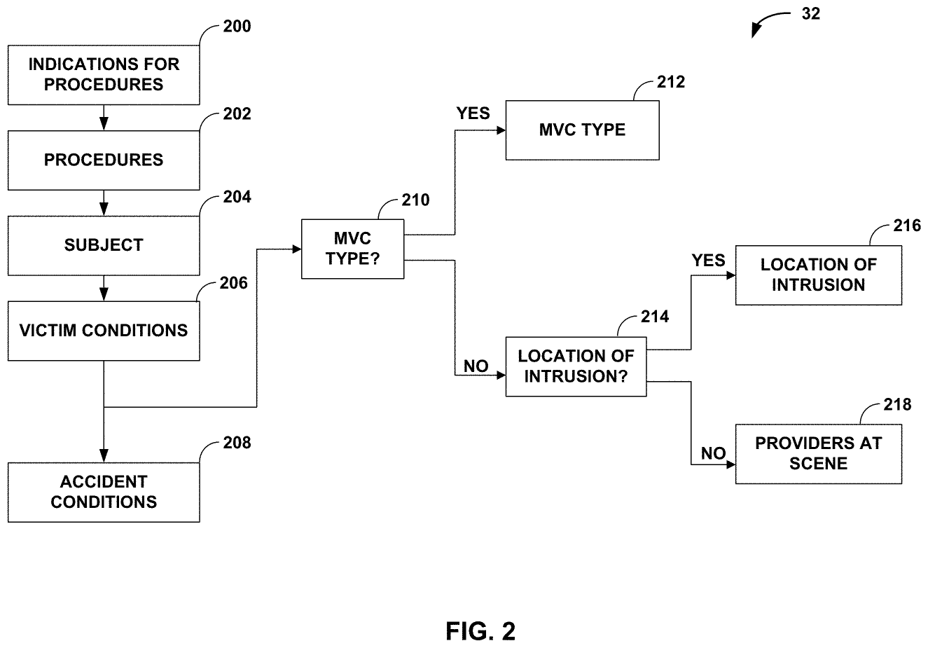

As shown in FIG. 2, the annotation schema 32 may include a set of annotation relationships describing how individual entities 200-218 are semantically connected with each other. For example, FIG. 2 depicts a portion of an example annotation schema 32 including a set of hierarchical relationships between various entities 200-218. Near the top of the hierarchy is an entity 200 representing any indications for procedures, for example, any verbal or written cues extracted from prehospital narrative records that directly or indirectly indicate any medical procedures that should be performed on a trauma victim. Underneath indications for procedures 200 are any entities directly indicative of medical procedures 202 that are being performed or should be performed on the trauma victim. Underneath procedures 202 are any entities indicative of the identity of the subject 204 (e.g., the trauma victim), such as their name, identifying features, their role in a traumatic incident, their position within a vehicle, etc. Underneath subject identity entities 204 are victim conditions 206, such as any keywords indicative of specific diagnosed injuries, traumas, or other conditions of subject 204.

In some examples, victim conditions 206 may include two or more sub-levels, such as accident conditions 208 (e.g., descriptions of the accident or scene of the trauma) as well as a binary indicator 210 of whether or not a specific category of motor-vehicle collision (MVC), (e.g., head-on collision, T-bone, sideswipe, etc.) is present within the prehospital narrative records. If there is an indication of MVC type (“yes” branch of 210), then MVC type entities 212 are listed as a sub-level of the binary indicator 210. If MVC type 212 is not indicated (“no” branch of 210), than another binary indication entity 214 is included as a sub-level of the binary indicator 210, wherein entity 214 includes a binary indication of whether any keywords are detected that describe a location of the intrusion or damage into the passenger compartment of the vehicle (e.g., a side of the vehicle that has been intruded upon). If such keywords are present (“yes” branch of 214), these keywords are included as a sub-level 216 of the binary indicator 214. If such keywords 216 are not present (“no” branch of 214), binary indicator 214 includes a sub-level of entities 218 including keywords indicative of any providers (EMS or other first responders) present at the scene of the traumatic incident.

In one non-limiting example, in the sentence: “Pt. (patient) was the driver of van that hit another vehicle head-on,” entities would be labeled for “pt.” as “subject” 204; “driver” as “driver/passenger status”; and “van that hit another vehicle head on” as “head-on” 212. Relationships would be added from the “pt.” to “driver” and from “driver” to “van that hit another vehicle head on” as the anchoring entity in the sentence.

In some examples, the keyword schema 32 may include six parent entities and 22 child entities. “MVC type” 212 may be added as an additional parent entity. Additional data gaps at the child entity level may include “insurance status,” “extrication time,” “cervical collar (absence),” “airway size,” “airway type,” and “number of attempts at a procedure.” “IV catheter placement” may be annotated as a procedure. In some examples, patients who are in severe MVC types 212 may nevertheless fail to meet the criteria of “head-on”, “T-bone”, or “rollover MVC”. Therefore an additional entity, “other severe”, may be added. As shown in Table 1, the final schema may include 7 parent entities and 30 child entities, totaling 37 entities. Each parent may have, for example, between 0 and 9 child entities. However, in some examples, the schema may include 47 total entities or more.

TABLE 1
Distribution of Annotation Entities Across EMS Documentation

| Entities | n (%) | |
|---|---|---|
| Total | 6364 | (100%) |
| Subject | 610 | (9.6%) |
| Negation | 78 | (1.2%) |
| EMS Run Number | 638 | (10.0%) |
| Victim Condition | | |
| Age | 317 | (5.0%) |
| Gender | 321 | (5.0%) |
| Insurance Status | 168 | (2.6%) |
| Driver Passenger Status | 260 | (4.1%) |
| Ejection from Car | 30 | (0.5%) |
| Seatbelt Presence | 209 | (3.3%) |
| Entrapment | 108 | (1.7%) |
| Extrication Time | 29 | (0.5%) |
| Accident Condition | | |
| Location of MVC | 159 | (2.5%) |
| Providers at Scene | 261 | (4.1%) |
| Triage | 31 | (0.5%) |
| Location of Intrusion | 158 | (2.5%) |
| Speed of Vehicle | 116 | (1.8%) |
| Severity of Intrusion | 117 | (2.8%) |
| Death on Scene | 8 | (0.1%) |
| Death in same | 1 | (0.02%) |
| compartment as driver | | |
| Airbag Presence | 192 | (3.0%) |
| MVC Type | | |
| Head-on | 27 | (0.4%) |
| T-bone | 30 | (0.5%) |
| Rollover | 48 | (0.8%) |
| Other Severe | 51 | (0.8%) |
| Other Minor | 40 | (0.6%) |
| Process Entity | | |
| Procedure | 497 | (7.8%) |
| Indication for Procedure | 1,256 | (19.7%) |
| Number of Attempts | 215 | (3.4%) |
| Size | 199 | (3.1%) |
| Airway Type | 36 | (0.6%) |
| Blood Products | 52 | (0.8%) |
| No Cervical Collar Placed | 11 | (0.2%) |
| End tidal CO₂ | 31 | (0.5%) |

Table 2 provides a summary of documentation practices and completeness for entities that may be required in prehospital admission data. These data elements may be required at the system level for all patients receiving prehospital admission care, and completeness may be assessed at the document-level for each patient.

TABLE 2
Distribution of Required Entities for Documentation

| | Number of | Percent of |
|---|---|---|
| | Notes with | Notes with |
| Entity | Documentation | Documentation |
| Subject | 151 | 96.8% |
| EMS Run Number | 156 | 100.00% |
| Age | 156 | 100.00% |
| Gender | 156 | 100.00% |
| Insurance Status | 151 | 96.8% |
| Passenger Status | 151 | 96.8% |
| Seatbelt Presence | 135 | 86.5% |
| Airbag Presence | 134 | 85.9% |
| MVC Type | 145 | 92.9% |
| Procedure - Intravenous Access | 137 | 87.9% |

Tables 3 and 4 provide a summary of documentation practices for entities of “indication for procedure” and “procedure”. Specifically, Table 3 shows the distribution of values for “indication for procedure” in prehospital admission data at one particular hospital.

TABLE 3
Distribution of values for Indication for Procedure entity

| Values | N | Annotation Frequency |
|---|---|---|
| Total | 1256 | 100% |
| Mental Status | 437 | 34.8% |
| Breath sounds and saturation | 646 | 51.4% |
| Pulse | 87 | 6.9% |
| Blood Pressure | 54 | 4.3% |
| Normal Vital Signs | 13 | 1.0% |
| Cardiac Arrest | 6 | 0.5% |
| Other | 13 | 1.0% |

Values may be grouped according to procedure indications. For example, patients with an “unresponsive” or “unconscious” mental status emergently require a procedure to secure their airway (i.e., endotracheal intubation). Attributes may be characterized for each procedural indication. For example, “unconsciousness” can be objectively demonstrated by a documented Glasgow Coma Scale score of 3, or it may be subjectively assessed. The two most represented values may include assessments of mental status and breath sounds (combined frequency 86.2%). Airway procedures were only documented with a frequency of 6.8% compared with a 77% frequency for documentation of vascular access procedures. Annotations for “indication were procedure” may be aggregated by notes and validated against aggregated procedure annotations to further understand the contrasting variations noted in documentation of indication for procedure and procedure performed. Table 4 shows the results of such an analysis.

TABLE 4
Distribution of values for Procedure entity

| Values | N | Annotation Frequency |
|---|---|---|
| Total | 497 | 100.0% |
| Vascular Access | 382 | 76.9% |
| Airway | 34 | 6.8% |
| Blood Infusion | 20 | 4.0% |
| Cardiopulmonary | 27 | 5.4% |
| Resuscitation (CPR) | | |
| Other procedure | 34 | 6.8% |

While there may be up to 437 annotations related to mental status, significant redundancy may be noted, with approximately three-to-four references to the patient's mental status within each note. 136 of 156 (87.2%) of notes may document a patient's mental status. Of notes with documentation, only 29 of 136 may carry the attribute for a “strong indication” for an airway procedure. For example, strong indications for an airway procedure may include “Glasgow Coma Scale (GCS) score <8”, “unresponsive”, “agonal respirations”, “unconscious”, and “declining mental status” or “an inability to protect one's airway”. While there may be 34 annotations related to an airway procedure, the majority of annotations may be redundant, with most patients having greater than four references to their mental status throughout the report. Additionally only 11 patients may have documentation of receiving a definitive airway despite 29 patients with a documented strong indication for emergent airway. This may be an important data gap identified in this analysis, as documentation of procedural indications and documentation of the reasons a procedure may not be performed (if indicated), may be essential for downstream performance monitoring and improvement efforts.

As shown in Table 5, documentation practices for clinically relevant mechanistic trauma triage criteria may also be analyzed. The presence in the note of any of these criteria may signal a high-impact MVC and escalate triage to a higher level of trauma care. Examples include highway vehicle speeds, patient ejection from the vehicle, severe (>12 inches) vehicle intrusion, death on the scene, roll-over or head-on MVCs. The frequency of these criteria within the corpus of data 12 may demonstrate the severity of MVCs included and could potentially correlate to procedure indications and procedure data for relevant patients.

TABLE 5
Distribution of mechanistic criteria across notes

| | # Notes with | % Notes with |
|---|---|---|
| Mechanistic Criteria | Documentation | Documentation |
| Death in same | 0 | 0.0% |
| compartment as passenger | | |
| Death on scene | 5 | 3.2% |
| Eject from car | 12 | 7.7% |
| Entrapment | 52 | 33.3% |
| Head-on collision | 23 | 14.7% |
| Rollover | 27 | 17.3% |
| Severity of Intrusion | 69 | 44.2% |
| Speed of Vehicle | 76 | 48.7% |

The developed schema may demonstrate wide variability in the range and completeness of documentation of clinically relevant entities within prehospital EMS MVC records. Using the NEMSIS 3 data standard, the schema may demonstrate that, while NEMSIS 3 has improved standardization and documentation of pre-hospital data, important gaps still exist. Specifically, an analysis of EMS records demonstrates the need for documentation standards of procedural indications including decisions to not perform an indicated procedure. With 1 in 5 patients suffering a preventable death, it is imperative to evaluate the role of the delivery of indicated or non-indicated procedures. It is likely that a key contributor to preventable deaths is the lack of delivering an indicated treatment or providing unnecessary treatments resulting in patient harm. Poor documentation of treatment indications hinders important performance monitoring and improvement efforts.

To illustrate this shortcoming, one may identify that 29 patients within a patient cohort had a strong medical indication for securing an emergent airway (i.e., intubation); however, only 11 patients may have had documentation of an airway procedure. There are two possible scenarios that can explain this discrepancy. First, the procedure could have been performed and not documented, or the patient may not have received an indicated procedure, thus creating an opportunity for EMS-provider education and training to improve performance and potentially improve rates of good clinical outcomes. By eliminating the first scenario through the development of more stringent data standards, increased quality-improvement and practice-improvement efforts can lead to improved clinical outcomes. Furthermore, NLP techniques may be used to harness and extract relevant information from free-text documentation 12.

While some data elements may be required for MVC patients in prehospital admission documentation, several of these elements may not complete for a particular corpus of data 12. While all patients in a corpus may have had documentation for “subject”, “EMS run number”, and “gender”, several other elements may not be completely documented at the document level. Rates for “seatbelt presence”, “airbag presence”, and “procedure—intravenous access” may be particularly low. This incompleteness in the data corpus 12 may indicate an opportunity to improve documentation rates through data standards and structured entry fields for required data elements.

A data corpus 12 may demonstrate language variation in several mechanistic triage elements. While head-on collisions and rollover collisions may be documented in 50 notes, there may be little standard language used to describe these accident conditions. For example, the following language may be used to describe rollover collisions in different reports: “went end-over-end 7 or 8 times;” “car possibly rolled;” “car on its side in the ditch.” These examples demonstrate that certainty factors into documentation of mechanistic criteria and that accident conditions may often be highly variable. Documentation of these mechanistic criteria may be vital to providing optimized patient treatment in a timely fashion. Additionally, mechanistic criteria may not be mutually exclusive. Structured guidelines for documenting the presence or absence of these criteria could lead to more complete documentation, however natural language processing (NLP) techniques may be key for utilizing highly variable English-language descriptors of these criteria. Further, standards should include the routine documentation (including documentation of absence) of mechanistic triage elements and improved information of vehicle safety equipment (such as the make and model of car seats), neither of which may typically be recommended or required data fields. More complete documentation of these elements will permit necessary research regarding vehicle safety design and potentially guide improved trauma triage activation and transfer protocols.

Additionally, the development of a schema may identify significant redundancy for documentation of certain data elements. This may be particularly prevalent for annotations relating to patient mental status and other indications for procedure. Reducing redundant or repetitive documentation may be important as EMS documentation is typically typed manually at the conclusion of an EMS transport. Eliminating redundant documentation and streamlining this process can reduce downtime between EMS runs, potentially allowing EMS providers to transport more patients per shift.

As prehospital data standardization becomes more widespread, it is imperative to maximize documentation of factors that may influence clinical outcomes. The development of an annotation schema may include analyzing prehospital EMS notes, characterizing the state of prehospital trauma information, and identifying gaps in process measure documentation. This work has potential to lead to more detailed and informed standards, improve prehospital data documentation, improve prehospital research and performance improvement efforts, and improve NLP techniques. This schema may need further refinement through the inclusion of multiple health systems' data for prehospital documentation and trauma information beyond MVC data. In some examples, the schema may utilize the present manually annotated gold standard schema to build and train NLP models to identify MVC elements in prehospital clinical documentation, and may be further expanded to create gold standards that include other traumatic-event information for downstream NLP model development.

Returning now to FIG. 1, system 10 may be configured to extract and analyze data according to the guidelines of a standardized keyword schema 32, as illustrated in the example of FIG. 2, above. System 10 includes NLP 14, configured to process the natural language of EMS prehospital records 12. While several off-the-shelf NLP algorithms and architectures already exist, in some examples in accordance with this disclosure, a second example keyword schema may support an improved NLP module 14 as an ensemble combination of other NLP systems. Additionally, another example keyword schema may be developed by using semantic models to test phrase synonymy. Both techniques may perform better than individual off-the-shelf NLP systems. Further improvement of the keyword schema used by processing system 10 may likely result from generalizing these techniques at scale by leveraging advanced semantic methodologies and deep-learning techniques. The improved keyword schemas may be developed according to the techniques of the following second example.

One example approach to bridge the shortcomings of existing EMS clinical reports 12 is the development of effective natural language processing (NLP) methods for these texts. Text-mining and NLP techniques hold promise for circumventing current limitations and the time-intensive nature of discrete element documentation or manual data abstraction of EMS reports by registrars. These techniques hold promise, since high-performing NLP systems may be able to automate or create significant efficiencies to the process of data abstraction from notes, reducing the effort required compared to manual data abstraction. The use of NLP for EMS may facilitate a data-driven approach for trauma research, performance monitoring, and improvement in patient outcomes.

Within the field of clinical NLP, no off-the-shelf solutions are available for the domain-specific language used in prehospital trauma surgery for named-entity recognition of key phrases. The efficacy of how existing systems can be best leveraged for notes with specific sublanguage characteristics is not well-characterized, and is the subject of this example.

The ability of several current clinical NLP tools and their ability to extract clinically relevant data elements as named entities and concepts may be examined within the domain of trauma surgery. A gold-standard corpus of manually annotated EMS trauma reports 12 of motor vehicle collisions (MVC) may be leveraged to develop an objective grading method for identifying the “best-at-task” system-annotation type for each named entity of interest.

The outputs of multiple annotation systems may be combined as an ensemble of NLP engines to improve performance over that provided by individual systems. These methods may also be extended to explore the use of semantic models to test word synonymy of clinically relevant phrases extracted from the best-at-task system annotations.

Clinically relevant phrases related to pre-hospital trauma medical care may be classified as named entities. Results may be derived through a detailed analysis of the performance of four widely-used clinical NLP annotation systems to achieve this goal, each on its own, and through a curated combination of system types (specific to NLP and clinical domain types used to label relevant constructs in text) as an ensemble of NLP engines.

The development of a gold-standard corpus may include of two main parts: (1) schema creation, and (2) manual text annotation. Entities of interest are shown in Table 6. An annotation schema may be created. Entities may be added iteratively to provide greater coverage and characterization of free-text documentation. The resulting annotation schema may also include guidelines for the annotation of associated temporal attributes, strength of Procedure Indication, and data hierarchy. Manual annotations may be made using the brat rapid annotation tool (BRAT).

TABLE 6
Example Annotation Guide Entries

Subject
Negation
EMS Run Number
Victim Condition
Age
Gender
Race
Insurance Status
Status
Ejection from Car
Seatbelt Presence
Entrapment
Extrication Time
Accident Condition
Location of MVC
Providers at Scene
Triage
Location of Intrusion
Speed of Vehicle
Severity of Intrusion
Death on Scene
Death in same compartment as driver
Airbag Presence
MVC Type
Head on
T-Bone
Rollover
Other Severe
Other Minor
Process Entity
Procedure
Procedure Indication
Number of Attempts
Size
Airway Type
Blood Products
No C-Collar Placed
End-Tidal Carbon Dioxide Level (ETCO2)

An initial annotation may be performed on two documents to determine annotation schema coverage. Following this step, 25 reports may be annotated (with 20% of documents overlapping across annotators) to establish inter-rater agreement (0.89 kappa, 99% agreement). Following this step, remaining reports may be manually annotated.

The Natural-Language Processing—Artifact Discovery and Preparation Toolkit (NLP-ADAPT) may be used, which provides a variety of software tools to annotate free-form medical texts and browse the output. Within NLP-ADAPT, these tools are designed to be interoperable using Apache's Unstructured Information Management Applications (UIMA) framework with the included annotator engines: Biomedical Information Collection and Understanding System (BioMedICUS), MetaMap, Apache clinical Text Analysis and Knowledge Extraction System (cTAKES), and the Clinical Language Annotation, Modeling, and Processing Toolkit (CLAMP); the annotation browser NLP Type and Annotation Browser (NLP-TAB); and the annotation compatibility engine, AMICUS Metasystem for Interoperation and Combination of UIMA Systems (AMICUS).

The gold standard annotations may be partitioned into two sets: 10 reports may be used to determine the “best-in-task” system annotations and the remaining 112 may be used for the performance evaluation of the selected best-at-task system annotations.

The 10 EMS reports used to determine best-at-task system annotations may be processed with the four NLP systems included in NLP-ADAPT. All generated system annotations may be compared against the gold standard annotation to characterize which system performed best-at-task for each target entity.

Methods may be developed in a JupyterLab Notebook for comparing BRAT annotation files to the XMI files produced by CLAMP, cTAKES, MetaMap. and BioMedICUS. The text span of each system annotation may be compared to the set of manual annotations by EMS report. Matches may be determined using the rules: a) having complete coverage (a system annotation was embedded within the span of the manual annotation or vice versa); b) having overlapping coverage (the system annotation's upper or lower bounds were outside either of the manual annotation's bounds or vice versa). As an example, the manual annotation, “ORIENTED-EVENT, ORIENTED-PERSON, ORIENTED-PLACE,” may be embedded in the system annotation “MENTAL STATUS: ORIENTED-EVENT, ORIENTED-PERSON, ORIENTED-PLACE, ORIENTED-TIME”, while the system annotation “AIRWAY IS PATENT, BREATHING IS” may overlap the manual annotation “AIRWAY IS PATENT” at its right boundary.

True positives (TP) may be defined as a match between system and manual annotation; false positives (FP) may be defined when a system annotation's span is not completely or partially covered by any manual annotation, and false negatives (FN) may be defined when a manual annotation's span is not covered by any system annotation. These values may then be used in a confusion matrix to calculate precision (positive predictive value), recall (sensitivity), and F1 score (harmonic mean of precision and recall) for each entity-and-system annotation pairing.

In some examples, no objective single measure may exist to characterize best-at-task annotation systems for entity capture in a complete way. Table 7 lists three examples in which measures that may be used to assess performance of best-at-task annotations on their own may have particular limitations. Example System Type 1 demonstrates that F1 score limitation may occur when a low denominator of total system annotations (n_{sys}) is present. In the present example, only 8 of 18 annotations may be identified; however, this annotation type may have the highest F1 score due to its low denominator (n_{sys}: 79 detected annotations). Example System Type 2 demonstrates that TP/TN ratio limitation may occur with complete annotation of a note (n_{sys}: 15,142). This may result in a high TP/FN ratio, but poor specificity. Example System Type 3 may perform the best overall: It may annotate all 18 entities with relatively good specificity (n_{sys}: 329).

TABLE 7
Limitations of individual metrics to evaluate
best-at-task annotation types.

| Example | | | | | | | |
|---|---|---|---|---|---|---|---|
| System | F₁ | | | | | | |
| Type | Score | TP/FN | TPN/√(n_{sys}) | TP | FN | FP | n_{sys} |
| 1 | 0.16 | 0.8 | 0.9 | 8 | 10 | 71 | 79 |
| 2 | 0.00 | 18.00 | 0.15 | 18 | 0 | 15124 | 15142 |
| 3 | 0.10 | 18.00 | 0.99 | 18 | 0 | 311 | 329 |

Abbreviations: TP, true positive; FN, false negative; FP, false positive; n_{sys}, total number of system annotations.

Qualitative evaluation for each annotation using NLP-TAB is a possible methodology for small sets. However, this method may not be feasible for the approximately 30 entity types needed to evaluate against each system. Furthermore, manual selection of the annotation types may be prone to subjective validation effects.

To develop and test an objective measure to help characterize best-at-task performance of off-the-shelf NLP systems, methods may be implemented in a JupyterLab Notebook that apply a rank across each system annotation type and entity using the following three objective measures of annotation performance:

$$\frac{T P}{F N} ;$$

F₁ score; and

$$\frac{T P}{\sqrt{{n}_{s y s}}} .$$

The geometric mean of each rank ordering may then be calculated to classify the best-at-task system annotation type for each entity. The system annotation type with the lowest geometric mean may be deemed the objective best-at-task system.

A merged set consisting of the top three ensemble best-at-task system annotations may be created using AMICUS on the remaining 112 EMS reports. The ability to select the system types for export into an AMICUS configuration file is provided by NLP-TAB. AMICUS may be used to merge the selected system annotations into a single XMI file per EMS report. The resulting set of system annotations may then be compared against the corresponding manually annotated set to assess the performance of the selected best-at-task system annotations.

As a proof of concept for validation of this method, the following two entities may be analyzed: “Procedure Indication” and “Severity of Intrusion”. The union of system annotations may be used as an ensemble to compare the spans of the merged annotations with those in the manually annotated documents to derive a percentage of coverage for each entity.

For Procedure Indication, a word2vec model, trained using the word2phrase method on electronic health record data, may be tested to determine how it affects recall. Some common phrases that may be identified during qualitative analysis of manually annotated records may include “unresponsive,” “unconscious,” “agonal,” “hypotensive,” “tachycardic,” “diminished breath sounds,” “absent breath sounds,” “desaturation,” “cardiopulmonary resuscitation (CPR),” “massive hemorrhage,” and “entrapment,”

To determine whether there is semantic synonymy or near-synonymy between the manually annotated EMS report and its associated system annotations, an experiment may be devised that compares these terms against the tokenized unigrams and bigrams from the set of manual and system annotations. Using the similarity method from the gensim implementation of word2vec, the cosine distance may be computed between the term of interest and the unigram or bigram phrase from the annotation as a measure of similarity. A threshold of cosine distance of 0.5 may be chosen after qualitatively evaluating several terms and their resultant set when processed through the word2vec distance function. Lists of those term/tokenized annotation pairings that have a cosine distance greater than the threshold may be saved for analysis. Further optimization of the ensemble system annotations may be obtained using the Levenshtein edit distance (LD) between the best-at-task list of each system annotation and the set of all gold standard annotations within each EMS report. Use of LD may be based on the premise that a lower LD correlates to synonymous and near-synonymous pairings. A semantic match may be assigned for the system/gold standard token pairing that gives the lowest LD value, which gives a baseline estimate for degree of synonymy of clinically significant tokenized phrases.

In some examples, 14 manual annotations from an initial set of 10 notes may pertain to the Severity of Intrusion entity. 42 manual annotations may pertain to the Procedure Indication entity. Three best-at-task system annotation types may be identified for each entity (Tables 8 and 9).

TABLE 8
Objective identification of three best-at-task annotation
systems for entity “Procedure Indication.”

| | | | | | Rank |
|---|---|---|---|---|---|
| System/Type | F₁ Score | TP | FN | FP | GeoMean |
| CLAMP Sentence | 0.03 | 21 | 21 | 1498 | 1.6 |
| cTAKES Sentence | 0.02 | 22 | 20 | 1869 | 2.2 |
| MetaMap Phrase | 0.01 | 25 | 17 | 3291 | 2.3 |

TABLE 9
Objective identification of thre best-at-task annotation
systems for entitiy “Severity of Intrusion.”

| | | | | | Rank |
|---|---|---|---|---|---|
| System/Type | F₁ Score | TP | FN | FP | GeoMean |
| CLAMP Sentence | 0.01 | 9 | 5 | 1510 | 1.0 |
| cTAKES Sentence | 0.00 | 4 | 10 | 1887 | 2.3 |
| CLAMP Chunk | 0.00 | 4 | 10 | 4501 | 3.0 |

The performance of the ensemble for the top 3 best-at-task systems may then be evaluated on the remaining 112 notes after processing them in AMICUS. 124 manual annotations pertaining to Severity of Intrusion may be noted in the 112 notes and the off-the-shelf performance of the three systems may then be evaluated. Individually, the three systems may perform relatively similarly, with 55%, 63%, and 68% annotation coverage, respectively, when compared with the set of gold standard annotations. The ensemble of all three systems may result in improved coverage (72%) (Table 10). Examples of phrases not annotated by any system may include: “major damage”, “6-12 inches”, “massive intrusion”, “pushed in 10-12 inches”, and “heavy damage with intrusion”.

TABLE 10
Performance of ensemble best-at-task systems for “Severity
of Intrusion” compared with gold standard.

| | Elements Annotated | Total Elements | Coverage |
|---|---|---|---|
| Best-at-task #1 alone | 78 | 124 | 63% |
| Best-at-task #2 alone | 84 | 124 | 68% |
| Best-at-task #3 alone | 68 | 124 | 55% |
| Combination | 89 | 124 | 72% |

931 manual annotations pertaining to Procedure Indication may be noted in the 112 notes and the off-the-shelf performance of the three systems may then be evaluated. Individually, the three systems may perform relatively similarly with 53%, 56%, and 64% annotation coverage, respectively, when compared with the gold standard. The ensemble of all three systems may result in improved coverage (68%) (Table 11). Examples of phrases not annotated by any system may include: “ANOx4” (alert and oriented 4-question scale), “GCS (Glasgow Coma Scale score) of 8,” “semi-conscious,” “mental status unresponsive,” “near unconscious,” “agonal resps.” (respirations).

TABLE 11
Performance of ensemble best-at-task systems for “Procedure
Indication” compared with gold standard.

| | Elements Annotated | Total Elements | Coverage |
|---|---|---|---|
| Best-at-task #1 alone | 931 | 524 | 56% |
| Best-at-task #2 alone | 931 | 596 | 64% |
| Best-at-task #3 alone | 931 | 495 | 53% |
| Combination | 931 | 634 | 68% |

To evaluate the degree of synonymy between the ensemble of best-at-task NLP systems and the gold standard annotations, common procedural indication phrases may be qualitatively characterized and best-at-task system and manual annotations may be processed using the word2phrase model (Table 12). Minimum cosine distance may be set to a threshold of 0.5. Finally, phrases identified in system and manual annotation may be matched based on the LD value. Of the 112 notes to be analyzed, 93% (104 of 112) coverage (match) may be identified for manual annotations. The mean LD value for matches may be, for example, 2.5 (range 0-19).

TABLE 12
Phrase2vec and system annotations.

| | | w2p common | Synonymous best-at-task |
|---|---|---|---|
| Note | w2p phrases | token | system annotation |
| 1 | breath sounds | equal | chest- lung sounds clear |
| | | bilaterally | and equal bilaterally |
| 2 | agonal | shallow | tachypnic shallow breathing |
| 3 | breath sounds | pulses | weak radial pulses |
| 4 | unconscious | unresponsive | mental status/unresponsive |
| 5 | tachycardic | lungs | lungs clear bilat |
| 6 | breath sounds | sounds | breath sounds- equal |
| 7 | tachycardic | sbp | 80's sbp. |
| 8 | unconscious | scene | alert on-scene |
| 9 | tachycardic | sats | initial spo2 sats |
| 10 | unconscious | regains | regains consciousness. |
| | | consciousness | |
| 11 | diminished | rhonchi | rhonchi/wheezing |
| | breath sounds | wheezing | |

Abbreviations: w2p, word2phrase; SBP, systolic blood pressure.

A novel approach may be developed to objectively classify best-at-task annotation systems for clinical NLP and characterize the performance of this technique through two specific clinical use cases. By leveraging a word2phrase model, the performance of these best-at-task annotation systems may be further optimized to obtain nearly 93% off-the-shelf performance for entity-labeling of key tokenized phrases.

While the ensemble of systems in NLP-ADAPT show promise in identifying key structural elements necessary for downstream identification of phrases spanning multiple tokens, the need to develop other pipeline components for named entity recognition is evident. Conversely, while the method of merging annotations from multiple UIMA systems for phrase extraction holds promise as a pipeline component for named entity recognition of phrases, it is also limiting in the following ways: MetaMap was developed in the domain of biomedical text retrieval, while cTAKES, BioMediCUS, and CLAMP were all designed to process standard clinical data. Thus, each system evaluated in this study was designed and trained on models for specific tasks outside the domain of trauma and pre-hospital medicine.

Given these constraints, other methods may be explored for identifying and extracting named phrase entities, including expansion of a test of synonymy through use of more sophisticated string-to-string alignment techniques to leverage the word2phrase model for automatically generating lexical patterns from the set of clinically relevant phrases for utilization of advanced information extraction methods or using deep networks for classification of target phrases akin to methods used for classification of entire sentences. Most importantly, a corpus of EMS trauma reports 12 may be created for building statistical models relevant to the domain of prehospital trauma medicine.

One limitation is that a limited number of entities (from Table 6) may initially be evaluated. Future examples should aim to examine more of these entities. Additionally, while some system-annotation types may score well using the geometric mean to identify best-at-task annotation systems, on examination, since the present method may be unable to provide lexical disambiguation of terms, there may be some misclassifications. One example is for the entity “Speed of Vehicle” where the system cTAKES may perform well with the “MedicationsMention” annotation type. On further examination, the terms that may provide a match are “speed” and “mph”, which may have different contextual meanings from those relating to a physical measurement of velocity. In this example, “speed” and “mph” are common street drugs. Thus, a human may be needed to be a final judge as to whether a particular annotation system type has been appropriately selected. Furthermore, the word2phrase model of this example may be trained on hospital data, and thus, while it may perform well for this example, it may fail on several prehospital terms. Furthermore, use of the Levenshtein edit distance (LD) measure may only account for synonymy between tokenized phrases, and thus may be prone to a possible increase in FN. Hence, methods for extracting relevant annotations based on clinically relevant phrases may need to be developed and evaluated. Lastly, the sample size of EMS reports 12 and associated annotations for this example is relatively small. Thus, meaningful results may require a scaled-up sample size of EMS reports 12.

This example describes a novel approach to characterize best-at-task annotation types when working across multiple NLP systems. Supplementing ensemble annotation systems with a word2phrase model may allow for over 90% entity capture for off-the-shelf systems across several terms of interest. While these results are encouraging, future work may be needed to evaluate these methods at the scale of thousands of EMS reports 12. Extending these methods and evaluating other methods for information extraction of clinically meaningful phrases or concepts within the domain of trauma medicine may be an ongoing process.

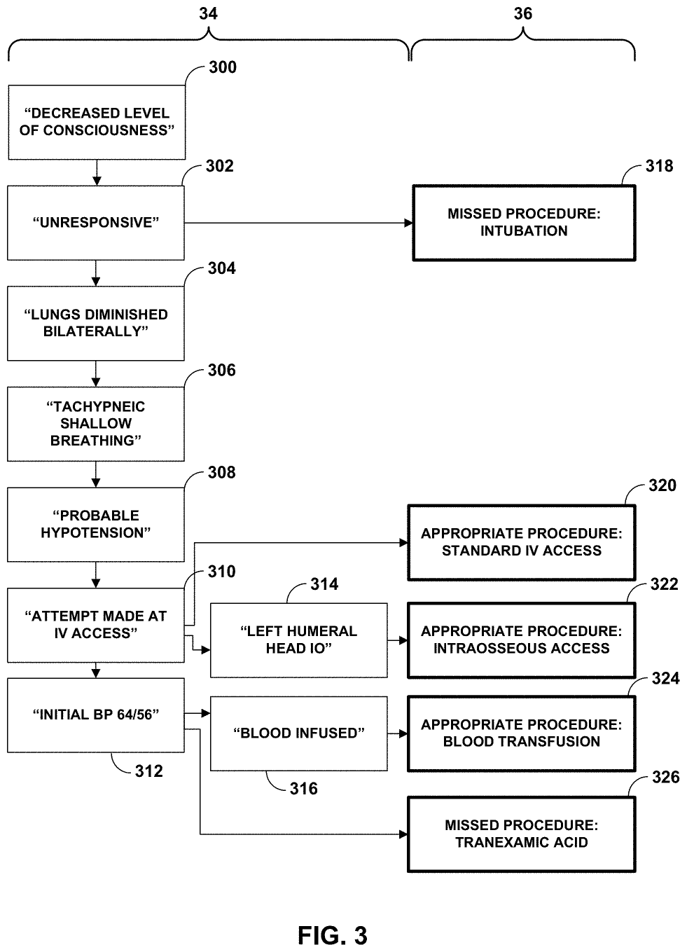

Returning again to FIG. 1, EMS data processing system 10 may be configured to extract and analyze key words from EMS data 12, for example, in accordance with any of the example keyword annotation schemas described above. For example, NLP 14 and IE 18 may be configured to extract and temporally arrange all clinically relevant entities to construct a timeline 34 (FIG. 3) of a trauma patient's prehospital clinical treatment.

Processing system 10 includes clinical procedure retriever 20. Clinical procedure retriever 20 is configured to determine, based on the entities extracted by NLP 14 and IE 18, a recommended treatment plan for each trauma patient. For example, clinical procedure retriever 20 may be configured to retrieve data 16 indicating an appropriate medical treatment corresponding to each of the trauma patient's symptoms or conditions, as indicated by the extracted entities.

Processing system 10 includes evaluation engine 22. Evaluation engine 22 is configured to compare the “recommended” treatments, as indicated by clinical procedure retriever 20, to the timeline of “applied” prehospital treatments. Evaluation engine 22 may be configured to determine, based on the comparison, a set 36 of procedural designations 318-326 (FIG. 3), including “appropriate” prehospital procedures, “inappropriate” (non-indicated) prehospital procedures, and/or “missed” (indicated but not performed) prehospital procedures. Some non-limiting examples of procedure indications (both applied and recommended) include airway procedures, intraosseous/intravenous access, blood transfusion, crystalloid bolus, LUCAS chest compressions, tranexamic acid, and needle decompression.

Processing system 10 includes report generator 24. Report generator 24 is configured to compile a report, based on the results of the comparison conducted by evaluation engine 22, evaluating the relative appropriateness or effectiveness of the applied prehospital treatments compared to the recommended prehospital treatments. Report generator 24 may output an indication of the evaluation, such as for display on screen 26. The evaluation report may be used for training or education, so as to improve future patient treatment and outcomes.

FIG. 3 is a flow diagram depicting an example process of extracting and evaluating clinical data from prehospital EMS records, in accordance with some techniques of this disclosure. The example flowchart of FIG. 3 is described with reference to system 10 of FIG. 1. System 10 may be configured to extract and temporally arrange all clinically relevant entities to construct a timeline 34 of a trauma patient's prehospital clinical treatments based on a set of extracted keywords 300-316 from the prehospital narrative records indicative of the prehospital patient conditions and/or clinical treatments. In the example shown in FIG. 3, system 10 has extracted and identified keywords including or indicative of a “decreased level of consciousness” 300; “unresponsive” 302; “lungs diminished bilaterally” 304; “tachypneic shallow breathing” 306; “probable hypotension” 308; “attempt made at IV access” 310; “initial BP” 312; “left humeral head IO” 314; and “blood infused” 316, and has arranged the keywords 300-316 vertically according to a chronological order of the diagnosed patient condition or performed procedures, as well as horizontally to indicate similar or related procedures (e.g., procedures 310, 314; procedures 312, 316), such as two or more individual procedures that are part of a larger procedure of medial treatment.

Evaluation engine 22 is configured to compare the “recommended” treatments, as indicated by clinical procedure retriever 20, to the timeline 34 of “applied” prehospital treatments 300-316. As shown in column 36, evaluation engine 22 may be configured to determine, based on the comparison, and output an indication of, a set of procedural designations 318-326, indicative of “appropriate” prehospital procedures 320-324, “inappropriate” (non-indicated) prehospital procedures (not shown in FIG. 3), and/or “missed” (indicated but not performed) prehospital procedures 318, 326. Some non-limiting examples of procedure indications (both applied and recommended) include airway procedures (e.g., intubation 318), intraosseous/intravenous access (310, 314), blood transfusion (316, 324), crystalloid bolus, LUCAS chest compressions, tranexamic acid (326), and needle decompression.

In the example shown in FIG. 3, evaluation engine 22 has determined, based on the “unresponsive” indication 302, and the lack of any follow-up procedure, that prehospital treatments failed to include a recommended “intubation” procedure 318. Similarly, evaluation engine 22 has determined, based on the “initial blood pressure” indication 312, that prehospital treatments failed to include a recommended “tranexamic acid” procedure 326.

As further shown in FIG. 3, evaluation engine 22 has determined, based on an “IV access” procedural indication 310, that prehospital treatments correctly included an appropriate “IV access” procedure 320. Similarly, evaluation engine 22 has determined, based on a “left humeral heat I/O” procedural indication 314, that prehospital treatments correctly included an appropriate “intraosseous access” procedure 322. Similarly, evaluation engine 22 has determined, based on a “blood infused” procedural indication 316, that prehospital treatments correctly included an appropriate “blood transfusion” procedure 320. Evaluation engine 22 may be configured to output for display an indication of the appropriate, inappropriate, and missed procedures.

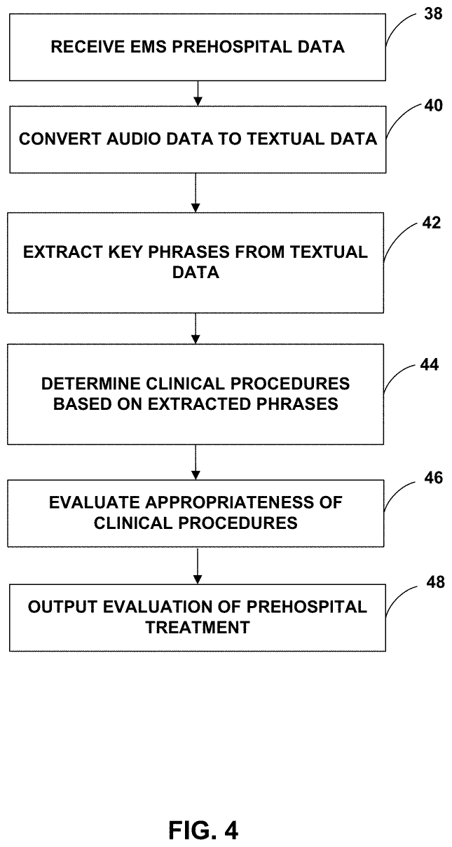

FIG. 4 is a flowchart illustrating an example operation in accordance with the present techniques. Specifically, FIG. 4 depicts a plurality of steps that may be performed by processing system 10 of FIG. 1. System 10 may receive EMS prehospital data 12 (38). EMS prehospital data 12 may include an EMT's narrative record, either in textual (written) or audio-recording form, indicating an evaluation and/or treatment of a trauma patient, such as a victim of a motor vehicle crash (MVC).

System 10 may process EMS data 12 according to a standardized keyword annotation schema. This data processing may include, for example, applying a speech-recognition module to convert audio data to textual data (40) and then run natural-language processing modules and information extraction modules to identify any clinically relevant key words or phrases, and then categorize them as “entities” according to the guidelines of the keyword annotation schema (42).

Based on the extracted entities (and their relationships under the schema), system 10 may construct a “treatment timeline” indicating a list of all clinical procedures and treatments applied to the trauma patient in the prehospital phase (44). System 10 may further compare the “applied” clinical procedures to a set of “recommended” clinical procedures as retrieved from storage in memory. Based on the comparison of the “applied” treatments to the “recommended” treatments, system 10 may evaluate the appropriateness of the “applied” treatments (46) and output an indication of the evaluation (48) for purposes of EMT training and improvement of patient outcomes.

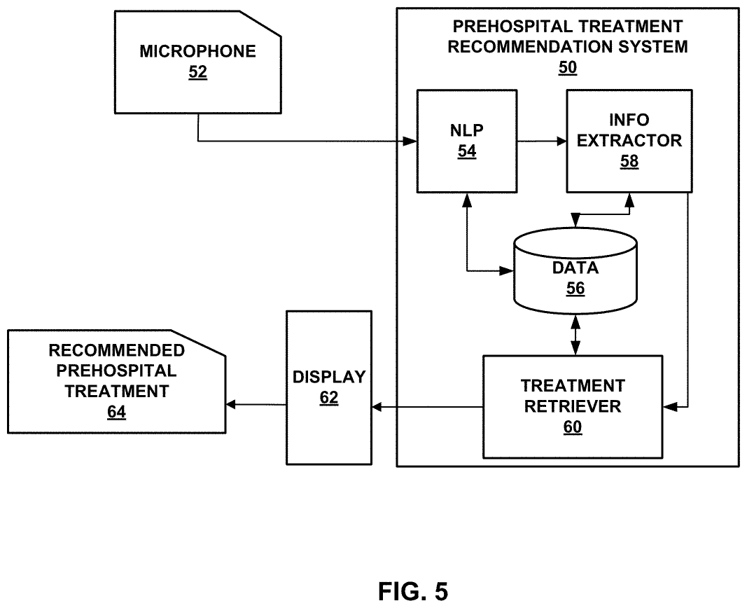

FIG. 5 is a block diagram illustrating an example computing system 50 for automatically generating real-time recommended treatments for trauma patients, in accordance with one or more techniques of the present disclosure. In the example of FIG. 5, system 50 may represent a computing device or computing system, such as a mobile computing device (e.g., a smartphone, a tablet computer, a personal digital assistant, and the like), a desktop computing device, a server system, a distributed computing system (e.g., a “cloud” computing system), or any other device capable of receiving audio data from microphone 52 and performing the techniques described herein.

As further described herein, system 50 is configured to receive data input, for example, audio data from microphone 52. Microphone 52 may, for example, be installed within an ambulance, and record an EMT's verbal, narrative assessment of one or more patients, such as a trauma patient.

System 50 is configured to receive audio data from microphone 52 and process the data according to a standardized keyword schema, such as any of the schema described with respect to FIG. 1, above. For example, system 50 may be configured to select one or more (e.g., a combination of multiple) speech-recognition modules, and apply the module(s) to the audio data to convert the audio signal, in real-time, to text-based data. NLP 54 and IE 58 may then be configured to identify and categorize clinically relevant keywords from the text-based data, in order to determine one or more symptoms or medical conditions of the patient.

System 50 includes treatment retriever 60. Treatment retriever 60 is configured to retrieve from memory 56, based on the determined symptom(s) or medical condition(s), a recommended therapy or treatment corresponding to the symptoms or conditions. System 50 may then output an indication of the recommended therapy or treatment, in real-time, to a monitor or screen visible by the EMT treating the patient, so that the EMT may apply the recommended therapy.

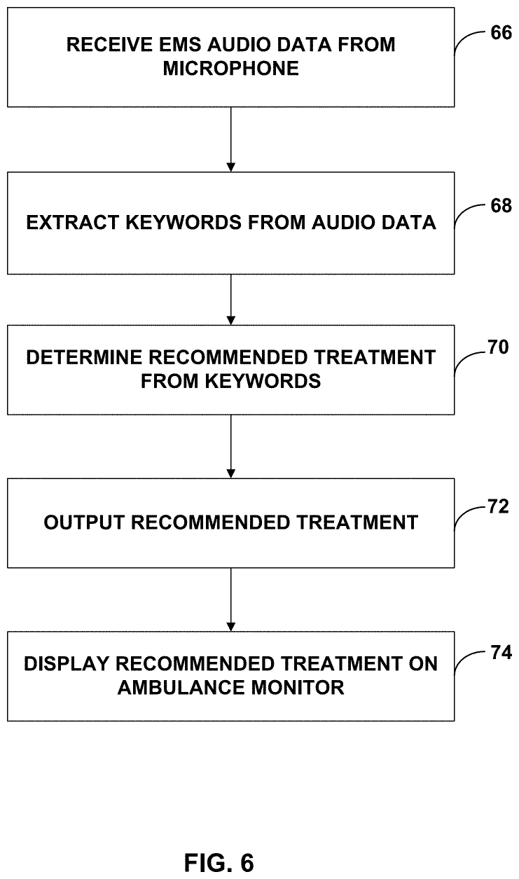

FIG. 6 is a flowchart illustrating an example operation in accordance with the present techniques. Specifically, FIG. 6 depicts a plurality of steps that may be performed by system 50 of FIG. 5. As further described herein, system 50 is configured to receive data input, for example, audio data from microphone 52 (66). Microphone 52 may, for example, be installed within an ambulance, and record an EMT's verbal, narrative assessment of one or more patients, such as a trauma patient.

System 50 is configured to receive audio data from microphone 52 and process the data according to a standardized keyword schema, such as any of the schema described with respect to FIG. 1, above (68). For example, system 50 may be configured to select one or more (e.g., a combination of multiple) speech-recognition modules, and apply the module(s) to the audio data to convert the audio signal, in real-time, to text-based data. System 50 may then be configured to identify and categorize clinically relevant keywords from the text-based data, in order to determine one or more symptoms or medical conditions of the patient.

System 50 may then retrieve from memory 56, based on the determined symptom(s) or medical condition(s), a recommended therapy or treatment corresponding to the symptoms or conditions (70). System 50 may then output an indication of the recommended therapy or treatment, in real-time, to a monitor or screen visible by the EMT treating the patient, so that the EMT may apply the recommended therapy (72, 74).

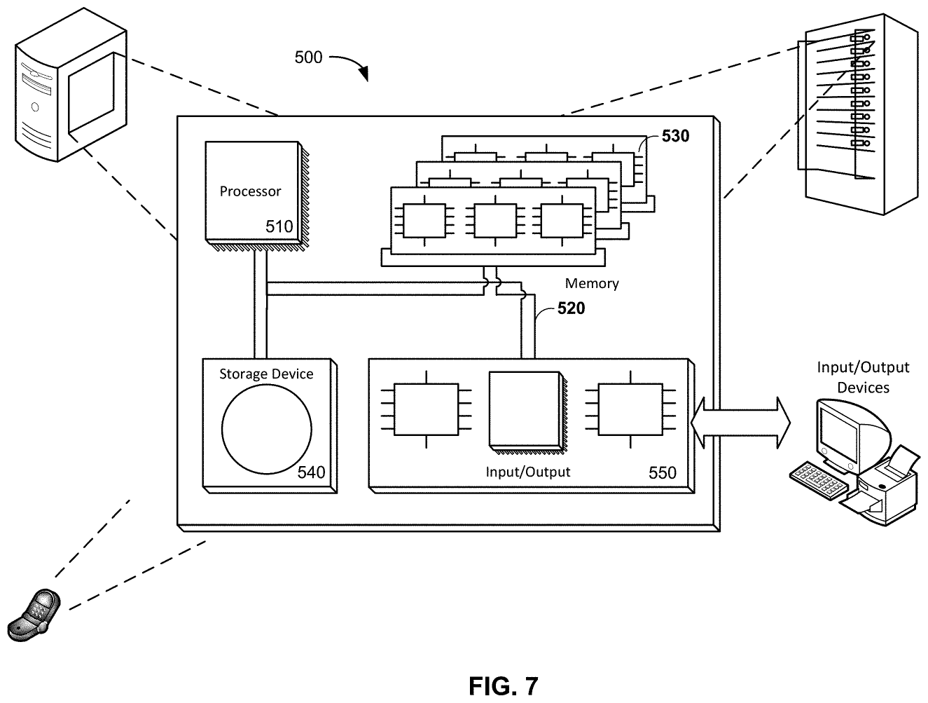

FIG. 7 is a block diagram illustrating an example of various devices that may be configured to implement one or more techniques of the present disclosure. That is, device 500 of FIG. 7 provides an example implementation for the EMS data processing system 10 of FIG. 1, or system 50 of FIG. 5, for processing EMS data. Device 500 may be a mobile device (e.g., a tablet, a personal digital assistant, or other mobile device), a workstation, a computing center, a cluster of servers, or other examples of a computing environment, centrally located or distributed, that is capable of executing the techniques described herein. Any or all of the devices may, for example, implement portions of the techniques described herein for generating and outputting predicted prostate cancer visualizations for display. In some examples, functionality of EMS data processing system 10 may be distributed across multiple computing devices, such as a cloud-based computing system for computing the predicted scores and generating the reports, and a client device, such as a table or mobile phone, for accessing and viewing the reports.

In the example of FIG. 7, computer-implemented device 500 includes a processor 510 that is operable to execute program instructions or software, causing the computer to perform various methods or tasks, such as performing the techniques for generating and/or using multiparametric models for prostate cancer prediction as described herein. Processor 510 is coupled via bus 520 to a memory 530, which is used to store information such as program instructions and/or other data while the computer is in operation. A storage device 540, such as a hard disk drive, nonvolatile memory, or other non-transient storage device stores information such as program instructions, data files of the multidimensional data and the reduced data set, and other information. The computer also includes various input-output elements 550, including parallel or serial ports, USB, Firewire or IEEE 1394, Ethernet, and other such ports to connect the computer to external devices such a printer, video camera, display device, medical imaging device, surveillance equipment or the like. Other input-output elements include wireless communication interfaces such as Bluetooth, Wi-Fi, and cellular data networks.

The computer itself may be a traditional personal computer, a rack-mount or business computer or server, or any other type of computerized system. The computer, in a further example, may include fewer than all elements listed above, such as a thin client or mobile device having only some of the shown elements. In another example, the computer is distributed among multiple computer systems, such as a distributed server that has many computers working together to provide various functions.

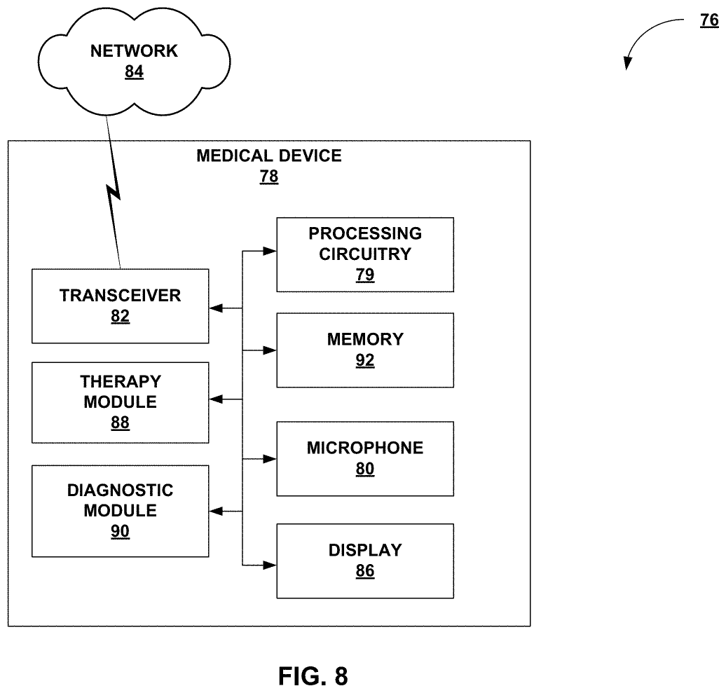

FIG. 8 is a block diagram depicting another example system 76 configured to implement the techniques of this disclosure. System 76 includes medical device 78 connected (e.g., wirelessly) to remote (e.g., cloud-based) data processing and storage network 84. Medical device 78 includes processing circuitry 79, microphone 80, transceiver 82, display screen 86, therapy module 88, diagnostic module 90, and memory 92. Processing circuitry 79 may include one or more processors or other circuitry configured to perform the functions attributed to medical device 78, such as controlling other components within medical device 78. Memory 92 may be configured to store data generated by any of the components, such as microphone 80, transceiver 92, therapy module 88, and/or diagnostic module 90.

Medical device 78 may be a self-contained, standalone machine or other device having a therapy module 88 and/or diagnostic module 90. For example, medical device 78 may include a therapy module 88 that includes a defibrillator, a LUCAS chest compressor, suction device, or other similar first-aid therapy module. Additionally or alternatively, medical device 78 may include a diagnostic module 90 such as an electroencephalogram (EEG) device, electrocardiogram (ECG) device, pulse oximeter, pulse rate monitor, blood pressure monitor, or other component configured to monitor one or more characteristics of a patient (e.g., one or more vital signs). In some examples, medical device 78 may be relatively mobile, such as housed within an ambulance or other first-aid vehicle or configured to be carried by a person.

Medical device 78 may include one or more microphone 80, configured to receive audio data in the immediate vicinity of a patient. For example, microphone 80 may record verbal descriptions from one or more first responders regarding a condition and/or a treatment of a patient.

Medical device 78 includes transceiver 82 configured to transmit the audio data from microphone 80 to cloud-based computing network 84. Cloud-based computing network 84 may be configured to first determine a preferred (e.g., superior in at least one aspect for the particular task) natural-language processing (NLP) engine, or in some examples, a particular combination of NLP engines, to apply to the audio data to extract one or more keywords from the audio data. Cloud-based computing network 84 may further determine, based on the extracted keywords, a set of associations or relationships between the keywords, and based on the keywords and their associations, determine a set of recommended therapies for the patient. Cloud-based computing network 84 may then transmit the set of recommended therapies back to transceiver 82, such that medical device 78 may display them on display screen 86. Attending first responders may then observe the recommended therapies on display screen 86, and apply them accordingly to the patient. In some examples, processing circuitry 79 may perform the functions described with respect to EMS data processing system 10 of FIG. 1 and/or prehospital treatment recommendation system 50 of FIG. 5.

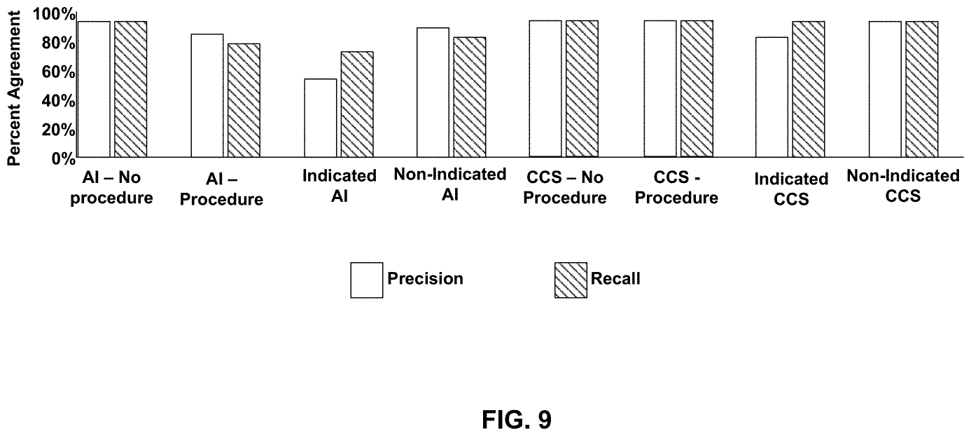

FIG. 9 is a bar graph depicting example precision and recall of natural-language processing compared with manual review for airway intervention (AI) and chest compression system (CSS), in accordance with the techniques of this disclosure. The data of FIG. 9 was generated using emergency medical services (EMS) data obtained for 22,529 patients between 2009-2018 in which the patients required scene transport to an American College of Surgeons or state designated trauma center following MVC. In one example, a previously validated ensemble NLP pipeline augmented with word2phrase embeddings, according to the techniques described above, has been used to characterize treatment indications and procedures for all patients. For NLP external validation, manual review of 243 records was performed by two trauma surgeons with Cohen's Kappa and percentage agreement computed for interrater reliability. The automated NLP results were compared to manual review. Precision and recall were calculated as measures of system performance, and the results are depicted in FIG. 9. Logistic regression was used to evaluate factors associated with treatments delivered.

As shown in FIG. 9, interrater reliability was high between manual reviewers with a percent agreement of 96.9% and a Cohen's kappa of 0.895. Precision and recall were overall high comparing the NLP system's determination for treatment indications and appropriateness to a manual review of prehospital treatments. Of the 22,529 patients, 936 (4.2%) had an indication for an airway intervention (AI), and 242 (1.1%) received an AI. 170 (0.8%) had an indication for intraosseous access (IO), and 110 (0.5%) received IO. 237 (1.1%) of patients had an indication for a crystalloid bolus (CB), and 319 (1.4%) received a CB. 157 (0.7%) of patients had an indication for chest compression system (CCS), and 53 (0.2%) received CCS. 55 (0.2%) had an indication for needle decompression (ND), and 21 (0.1%) received ND. Patients treated by paramedics (vs EMT) trended towards receiving more indicated airway procedures (OR 2.02, 95% CI 0.92-4.46, p=0.08). The interaction of multiple (vs one) injured patients at the scene with treatment by a paramedic (vs EMT) was associated with an OR 2.6 (95% CI 0.99-6.7, p=0.051) to receive indicated treatment; however, did not reach statistical significance. Based on the high precision and recall resulting from the system applying the NLP techniques to EMS data, the systems described herein may be used for review of EMS service and/or real-time recommendations of treatments for patients based on EMS data.

The following numbered clauses provide some examples of this disclosure:

Clause 1: In some examples, a system includes a data repository configured to store a plurality of treatments and a plurality of respective clinically relevant phrases, each of the respective clinically relevant phrases being associated with at least one treatment of the plurality of treatments; and a computing system comprising processing circuitry configured to: receive prehospital data; determine at least one clinically relevant phrase of the plurality of clinically relevant phrases present in the EMS prehospital data; and determine at least one recommended treatment associated with the at least one clinically relevant phrase.

Clause 2: In some examples of the system of clause 1, the processing circuitry is configured to determine the at least one clinically relevant phrase by at least categorizing the EMS prehospital data according to a keyword annotation schema.

Clause 3: In some examples of the system of clause 2, the keyword annotation schema comprises a plurality of entities.

Clause 4: In some examples of the system of clause 3, the entities include at least one of: a subject; a negation; an EMS run number; a victim condition; an accident condition; a speed of a vehicle; a collision type; or a process entity.

Clause 5: In some examples of the system of any of clauses 1-4, the EMS prehospital data includes audio data, and the processing circuitry is further configured to perform speech recognition that converts the audio data to textual data.

Clause 6: In some example of the system of any of clauses 1-5, the processing circuitry is further configured to: determine one or more applied procedural indications from the EMS prehospital data; compare the at least one recommended treatment to the applied procedural indications; and output an indication of the comparison.

Clause 7: In some examples of the system of clause 6, the one or more applied procedural indications includes at least one of: an airway procedure; intraosseous or intravenous access; a blood transfusion; a crystalloid bolus; LUCAS chest compressions; tranexamic acid; or a needle decompression.

Clause 8: In some examples of the system of clause 6, the indication of the comparison comprises at least one of: an appropriate procedure; an indicated-but-not-performed procedure; and a non-indicated procedure.

Clause 9: In some examples of the system of any of clauses 1-8, the computing system includes one or more of a cloud-based computing platform, a mobile device, a laptop, or a server.

Clause 10: In some examples of the system of any of clauses 1-9, the processing circuitry is configured to output the recommended treatment to a user via a user interface.

Clause 11: In some examples, a method includes: receiving, by processing circuitry, prehospital data; determining, by the processing circuitry, at least one clinically relevant phrase present in the EMS prehospital data; determining, by the processing circuitry, at least one recommended treatment associated with the at least one clinically relevant phrase; and outputting, by the processing circuitry, the at least one recommended treatment.

Clause 12: In some examples of the method of clause 11, the EMS prehospital data comprises audio data, and the method further includes: performing speech recognition to convert the audio data to textual data.

Clause 13: In some examples of the method of clause 11 or clause 12, the method further includes: determining one or more applied procedural indications from the data; comparing the recommended treatment to the applied procedural indications; and outputting an indication of the comparison.

Clause 14: In some examples of the method of clause 13, the one or more applied procedural indications include at least one of: an airway procedure; intraosseous or intravenous access; a blood transfusion; a crystalloid bolus; LUCAS chest compressions; tranexamic acid; or a needle decompression.

Clause 15: In some examples of the method of clause 13, the indication of the comparison includes at least one of: an appropriate procedure; an indicated-but-not-performed procedure; or a non-indicated procedure.

Clause 16: In some examples of the method of clause 13, the indication of the comparison comprises a statistical report indicating an evaluation of the one or more applied procedural indications.

Clause 17: In some examples of the method of clause 13, the method further includes temporally relating the procedural indications to construct a clinical procedure timeline.

Clause 18: In some examples of the method of any of clauses 11-17, outputting the at least one recommended treatment comprises outputting the recommended treatment to a user via a user interface.

Clause 19: In some examples of the method of any of clauses 11-18, the EMS prehospital data includes EMS technician textual notes.

Clause 20: IN some examples of the method of any of clauses 11-19, determining the at least one clinically relevant phrase present in the EMS prehospital data includes processing, by the processing circuitry, the EMS prehospital data according to a keyword annotation schema; and the method further includes selecting at least one natural-language-processing (NLP) engine.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, include compact discs (CDs), laser discs, optical discs, digital versatile discs (DVDs), floppy disks and Blu-ray discs, where “disks” usually reproduce data magnetically, while “discs” reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Further examples are provided in the Appendix attached below and incorporated herein by reference.

What is claimed is:

1. A system comprising:
a data repository configured to store a plurality of treatments and a plurality of respective clinically relevant phrases, each of the respective clinically relevant phrases being associated with at least one treatment of the plurality of treatments; and
a computing system comprising processing circuitry configured to:
receive prehospital data;
determine at least one clinically relevant phrase of the plurality of clinically relevant phrases present in the EMS prehospital data; and
determine at least one recommended treatment associated with the at least one clinically relevant phrase.

2. The system of claim 1, wherein the processing circuitry is configured to determine the at least one clinically relevant phrase by at least categorizing the EMS prehospital data according to a keyword annotation schema.

3. The system of claim 2, wherein the keyword annotation schema comprises a plurality of entities.

4. The system of claim 3, wherein the entities comprise at least one of:
a subject;
a negation;
an EMS run number;
a victim condition;
an accident condition;
a speed of a vehicle;
a collision type; or
a process entity.

5. The system of claim 1, wherein the EMS prehospital data comprises audio data, and wherein the processing circuitry is further configured to perform speech recognition that converts the audio data to textual data.

6. The system of claim 1, wherein the processing circuitry is further configured to:
determine one or more applied procedural indications from the EMS prehospital data;
compare the at least one recommended treatment to the applied procedural indications; and
output an indication of the comparison.

7. The system of claim 6, wherein the one or more applied procedural indications comprises at least one of:
an airway procedure;
intraosseous or intravenous access;
a blood transfusion;
a crystalloid bolus;
LUCAS chest compressions;
tranexamic acid; or
a needle decompression.

8. The system of claim 6, wherein the indication of the comparison comprises at least one of:
an appropriate procedure;
an indicated-but-not-performed procedure; and
a non-indicated procedure.

9. The system of claim 1, wherein the computing system comprises one or more of a cloud-based computing platform, a mobile device, a laptop, or a server.

10. The system of claim 1, wherein the processing circuitry is configured to output the recommended treatment to a user via a user interface.

11. A method comprising:
receiving, by processing circuitry, prehospital data;
determining, by the processing circuitry, at least one clinically relevant phrase present in the EMS prehospital data;
determining, by the processing circuitry, at least one recommended treatment associated with the at least one clinically relevant phrase; and
outputting, by the processing circuitry, the at least one recommended treatment.

12. The method of claim 11, wherein the EMS prehospital data comprises audio data, and wherein the method further comprises:
performing speech recognition to convert the audio data to textual data.

13. The method of claim 11, further comprising:
determining one or more applied procedural indications from the data;
comparing the recommended treatment to the applied procedural indications; and
outputting an indication of the comparison.

14. The method of claim 13, wherein the one or more applied procedural indications comprise at least one of:
an airway procedure;
intraosseous or intravenous access;
a blood transfusion;
a crystalloid bolus;
LUCAS chest compressions;
tranexamic acid; or
a needle decompression.

15. The method of claim 13, wherein the indication of the comparison comprises at least one of:
an appropriate procedure;
an indicated-but-not-performed procedure; or
a non-indicated procedure.

16. The method of claim 13, wherein the indication of the comparison comprises a statistical report indicating an evaluation of the one or more applied procedural indications.

17. The method of claim 13, further comprising temporally relating the procedural indications to construct a clinical procedure timeline.

18. The method of claim 11, wherein outputting the at least one recommended treatment comprises outputting the recommended treatment to a user via a user interface.

19. The method of claim 11, wherein the EMS prehospital data comprises EMS technician textual notes.

20. The method of claim 11, wherein determining the at least one clinically relevant phrase present in the EMS prehospital data comprises processing, by the processing circuitry, the EMS prehospital data according to a keyword annotation schema; and wherein the method further comprises selecting at least one natural-language-processing (NLP) engine.
